(19) United States
(12) Patent Application Publication
(10) Pub. No.: US 2017/0319808 A1
(43) Pub. Date: Nov. 9, 2017

(54) PATIENT INTERFACE DEVICE WITH DRAWSTRING ADJUSTMENT

(71) Applicant: KONINKLIJKE PHILIPS N.V., Eindhoven (NL)

(72) Inventor: MATTHEW ANSWINE, APOLLO, PA (US)

(21) Appl. No.: 14/916,206

(22) PCT Filed: Sep. 4, 2014
(86) PCT No.: PCT/IB2014/064242

§ 371 Date: Mar. 3, 2016

Related U.S. Application Data
(60) Provisional application No. 61/873,427, filed on Sep. 4, 2013.

(51) Int. Cl. A61M 16/06

(52) U.S. Cl. CPC A61M 16/0666; A61M 2210/0618

(57) ABSTRACT

A patient interface device includes a patient sealing assembly having a cushion member and a headgear assembly for securing the patient sealing assembly to a head of the patient. The headgear assembly has a first drawstring, a second drawstring and a locking member structured to be positioned on a top of the head of the patient responsive to the patient interface device being donned by the patient. The first drawstring has a first distal end coupled to a first side of the patient sealing assembly and a first proximal end received through the locking member, and the second drawstring has a second distal end coupled to a second side of the patient sealing assembly and a second proximal end received through the locking member, wherein a fit of the patient interface device may be selectively adjusted using the first drawstring, the second drawstring and the locking member.

### CROSS-REFERENCE TO RELATED APPLICATIONS

This patent application claims the priority benefit under 35 U.S.C. §119(e) of U.S. Provisional Application No. 61/873,427 filed on Sep. 4, 2013, the contents of which are herein incorporated by reference.

### BACKGROUND OF THE INVENTION

### 1. Field of the Invention

The present invention pertains to patient interface devices structured to deliver a flow of breathing gas to a user, and, in particular, to a patient interface device having a headgear that employs a drawstring mask adjustment mechanism.

### 2. Description of the Related Art

There are numerous situations where it is necessary or desirable to deliver a flow of breathing gas non-invasively to the airway of a patient, i.e., without intubating the patient or surgically inserting a tracheal tube into the patient's esophagus. For example, it is known to ventilate a patient using a technique known as non-invasive ventilation. It is also known to deliver positive airway pressure (PAP) therapy to treat certain medical disorders, the most notable of which is OSA. Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible sealing cushion on the face of a patient. The mask component may be, without limitation, a nasal mask that covers the patient's nose, a nasal/oral mask that covers the patient's nose and mouth, a nasal cushion that rests beneath the patient's nose (such as a “pillows” style nasal cushion having nasal prongs that are received within the patient's nares or a “cradle” style nasal cushion that rests beneath and covers the patient's nares) or a full face mask that covers the patient's face. Such patient interface devices may also employ other patient contacting components, such as forehead supports, cheek pads and chin pads. The sealing cushion typically has a support portion coupled to a sealing portion, which may integrated together as a single part or that may be separate components that when combined together in the final assembly provide the sealing and support functions. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

In designing patient interface devices, particularly those for pressure support therapy for treating OSA, accurate fit to a given patient is a critical measure of therapy success. Poor fit to a given patient can result in excessive mask leak (reduced therapy pressures) and/or mask over tightening (which can lead to development of undesirable sores, abrasions and/or indentations on the patient's face). Specifically, poor fit of headgear impacts therapy success in that comfort and seal are compromised.

### SUMMARY OF THE INVENTION

In one embodiment, a patient interface device is provided that includes a patient sealing assembly having a cushion member for delivering a flow a breathing gas to a patient and a headgear assembly for securing the patient sealing assembly to a head of the patient. The headgear assembly has a first drawstring, a second drawstring and a locking member structured to be positioned on a top of the head of the patient responsive to the patient interface device being donned by the patient. The first drawstring has a first distal end coupled to a first side of the patient sealing assembly and a first proximal end received through the locking member, and the second drawstring has a second distal end coupled to a second side of the patient sealing assembly and a second proximal end received through the locking member, wherein a fit of the patient interface device may be selectively adjusted using the first drawstring, the second drawstring and the locking member.

In another embodiment, a method of delivering a flow of breathing gas to a patient is provided. The method includes donning a patient interface device as described above, adjusting the fit of the patient interface device using the first drawstring, the second drawstring and the locking member, and delivering the flow of breathing gas to the patient through the patient sealing assembly.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a system adapted to provide a regimen of respiratory therapy to a patient according to one exemplary embodiment;

FIG. 2 is a front isometric view of a frame member forming a part of a patient interface device of the system of FIG. 1; and

FIG. 3 is a top plan view of a headgear assembly forming a part of a patient interface device of the system of FIG. 1.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

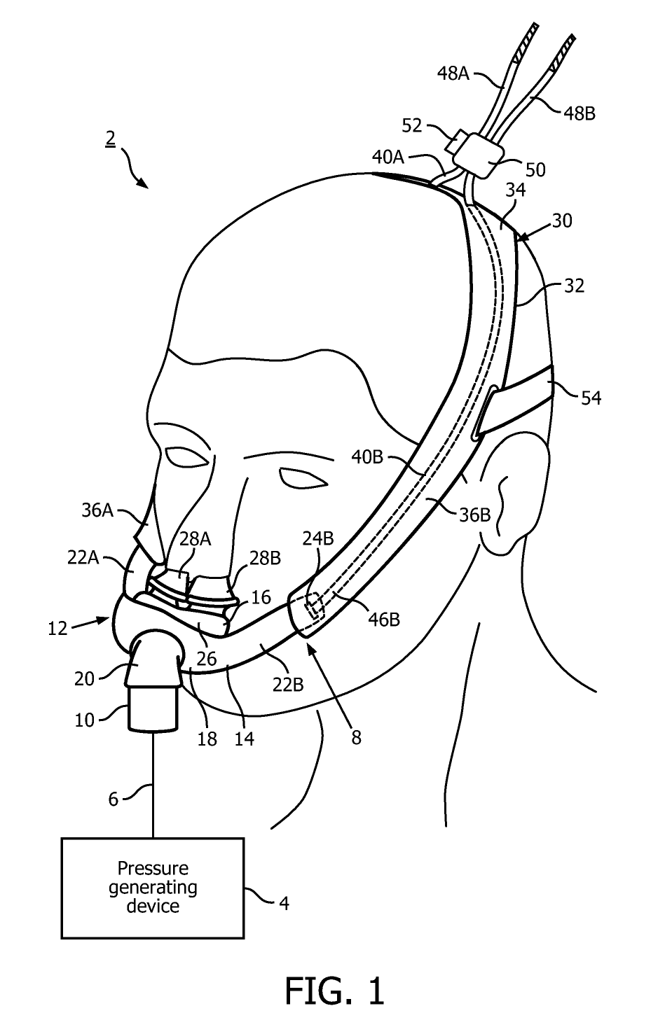

A system 2 adapted to provide a regimen of respiratory therapy to a patient according to one exemplary embodiment is generally shown in FIG. 1. System 2 includes a pressure generating device 4, a delivery conduit 6, and a patient interface device 8 having a fluid coupling conduit 10. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8 through fluid coupling conduit 10, which in the illustrated embodiment is an elbow connector. Delivery conduit 6 and patient interface device 8 are often collectively referred to as a patient circuit.

In the exemplary embodiment, patient interface 8 includes a patient sealing assembly 12, which in the illustrated embodiment is a nasal mask. However, other types of patient sealing assemblies, such as, without limitation, a nasal/oral mask or a full face mask, which facilitate the delivery of the flow of breathing gas to the airway of a patient, may be substituted for patient sealing assembly 12 while remaining within the scope of the present invention. Patient sealing assembly 12 includes a frame member 14 having a cushion member 16 coupled thereto, each of which is described in greater detail below.

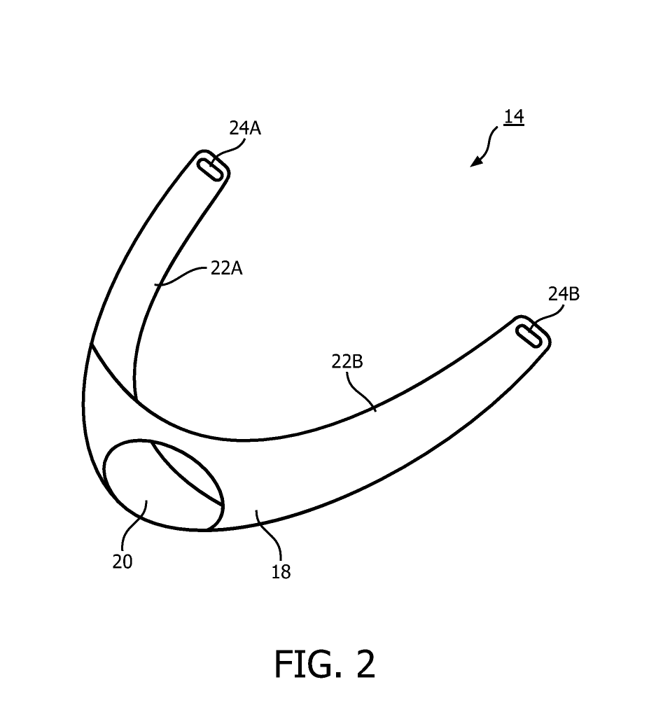

FIG. 2 is a front isometric view of frame member 14 according to the exemplary embodiment. In the illustrated embodiment, frame member 14 is made of a rigid or semi-rigid material, such as, without limitation, an injection molded thermoplastic or silicone. Frame member 14 includes a connecting portion 18 having a central annular opening 20. Frame member 14 further includes arms 22A and 22B which extend outwardly from connecting portion 18. Each arm 22A, 22B includes a respective slot 24A, 24B provided in the distal end thereof. The function of the slots 24A, 24B is described below.

As seen in FIG. 1, both fluid coupling conduit 10 and cushion member 16 are received by and supported by connecting portion 18. More specifically, fluid coupling conduit 10 is coupled to a conduit side of connecting portion 18 through opening 20, and cushion member 16 is coupled to a cushion side of connecting portion 18 in a manner which surrounds opening 20. This configuration allows the flow of breathing gas from pressure generating device 4 to be communicated to cushion member 16, and then to the airway of a patient.

In the illustrated embodiment, cushion member 16 is a pillows style nasal cushion. Cushion member 16 is made of flexible, cushiony, elastomeric material, such as, without limitation, silicone, an appropriately soft thermoplastic elastomer, a closed cell foam, or any combination of such materials. Cushion member 16 includes a main body portion 26 that is structured to be fluidly coupled to coupling conduit 10 through connecting portion 18 of frame member 14. Cushion member 16 further includes nasal prongs 28A and 28B which extend from a top side of main body portion 26. Nasal prongs 28A and 28B are structured to be received within the nares of the patient for delivering a breathing gas to the airways of the patient through the nares.

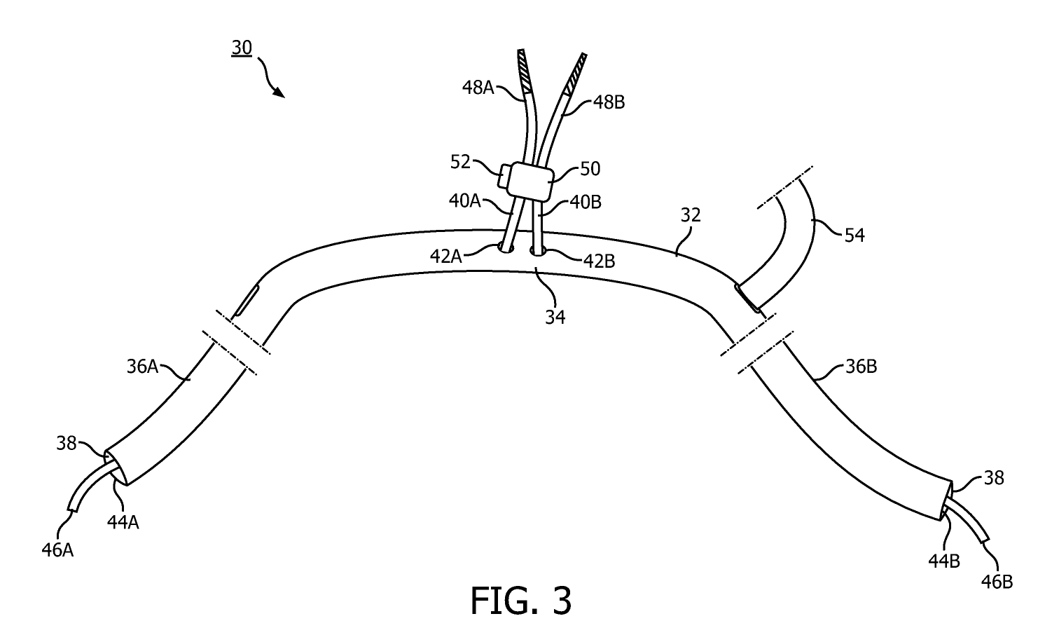

As seen in FIG. 1, patient interface 8 further includes a headgear assembly 30 that is structured to couple patient sealing assembly 12 to the head of the patient. FIG. 3 is a top plan schematic diagram of headgear assembly 30. Headgear assembly 30 includes a sleeve member 32 having a central portion 34 configured to rest on top of the patient's head, and first and second side members 36A and 36B each configured to extend along a respective side of patient's head. In the exemplary embodiment, sleeve member 32 is made of a fabric material, such as, without limitation, cotton or polyester. Sleeve member 32 defines an internal channel or chamber which houses at least a part of first and second drawstrings 40A, 40B.

More particularly, first drawstring 40A is inserted into a hole 42A provided in central portion 34 and extends along a length of first side member 36A to an opening 44A which allows a distal end 46A of first drawstring 40A to exit channel 38. Similarly, second drawstring 40B is inserted into a hole 42B provided in central portion 34 and extends along a length of second side member 36B to an opening 44B which allows a distal end 46B of second drawstring 40B to exit channel 38. The proximal ends 48A, 48B of first and second drawstrings 40A and 40B are received through the central opening of a spring loaded cord lock member 50 adjacent central portion 34. Cord lock member 50 has an actuator 52 which is configured to open and close cord lock member 50 (i.e., it is configured to control the spring action thereof). Finally, in the exemplary embodiment, headgear assembly 30 includes an adjustable rear strap member 54 structured to extend from second side member 36B to first side member 36A at the rear of the patient's head.

As seen in FIG. 1, in order to assemble patient interface device 8 for use, distal end 46A of first drawstring 40A is fixedly attached (using any suitable means, such as, without limitation, a secure knot) to arm 22A of frame member 14 through slot 24A and distal end 46B of second drawstring 40B is fixedly attached (using any suitable means, such as, without limitation, a secure knot) to arm 22B of frame member 14 through slot 24B. Patient interface device 8 may then be donned by the patient as shown in FIG. 1 in a manner wherein patient sealing assembly 12 sealingly engages the nares of the patient.

In operation, in order to adjust the fit of patient interface device 8 once donned, the patient merely needs to squeeze actuator 52 of cord lock member 50 and pull the proximal ends 48A, 48B of first and second drawstrings 40A, 40B while sliding cord lock member 50 until the desired tightness is achieved. Such an adjustment mechanism is simpler than current headgear designs, which typically employ multiple tabs and fasteners (e.g., Velcro®), which can wear out, become misaligned and/or pull on the patient's hair. In contrast, the adjustment of the present invention via the drawstring mechanism as described is simpler than current designs and, due to such simplicity, may benefit patient's having limited dexterity.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device, comprising:
a patient sealing assembly having a cushion member for delivering a flow a breathing gas to a patient, a first attachment portion and a second attachment portion; and
a headgear assembly for securing the patient sealing assembly to a head of the patient, the headgear assembly having a first drawstring, a second drawstring and a locking member structured to be positioned on a top of the head of the patient responsive to the patient interface device being donned by the patient, the first drawstring having a first distal end fixedly attached the first attachment portion and a first proximal end received through the locking member, the second drawstring having a second distal end fixedly attached the second attachment portion and a second proximal end received through the locking member, wherein the headgear assembly includes a sleeve member extending from a first side of the patient sealing portion configured to rest on the top of the head of the patient responsive to the patient interface device being donned by the patient, and first and second side members each being configured to extend along a respective side of the head responsive to the patient interface device being donned by the patient, wherein the first attachment portion is received within an end of the first side member and the first drawstring extends within the first side member along a length of the first side member to the central portion and the second attachment portion is received within and end of the second side member and the second drawstring extends within the second side member along a length of the second side member to the central portion, and wherein a fit of the patient interface device may be selectively adjusted using the first drawstring, the second drawstring and the locking member.

2. (canceled)

3. The patient interface device according to claim 1, wherein the first and second proximal ends are passed through one or more holes provided in the central portion.

4. The patient interface device according to claim 1, wherein the sleeve member comprises a fabric material.

5. The patient interface device according to claim 1, wherein the cushion member comprises a nasal mask.

6. The patient interface device according to claim 5, wherein the cushion member comprises a nasal cushion.

7. The patient interface device according to claim 5, wherein the cushion member comprises a pillows style nasal cushion having first and second nasal prongs.

8. The patient interface device according to claim 1, wherein the patient sealing assembly includes a frame member coupled to the cushion member, the frame member having a first arm that includes the first attachment portion at an end thereof and a second arm that includes the second attachment portion at an end thereof.

9. A method of delivering a flow of breathing gas to a patient, comprising:
donning a patient interface device including a patient sealing having a cushion member, a first attachment portion and a second attachment portion, and a headgear assembly having a first drawstring, a second drawstring and a locking member in a manner wherein the locking member is positioned on a top of the head of the patient, the first drawstring having a first distal end fixedly attached to the first attachment portion and a first proximal end received through the locking member, the second drawstring having a second distal end fixedly attached to the second portion and a second proximal end received through the locking member, wherein the headgear assembly includes a sleeve member extending from a first side of the patient sealing assembly to a second side of the patient sealing assembly, the sleeve member having a central portion configured to rest on the top of the head of second side members each being configured to extend along a respective side of the head responsive to the patient interface device being donned by the patient, wherein the first attachment portion is received within an end of the first side member and the first drawstring extends within the first side member along a length of the first side member to the central portion and the second attachment portion is received within an end of the second side member and the second drawstring extends within the second side member along a length of the second side member to the central portion;
adjusting a fit of the patient interface device using the first drawstring, the second drawstring and the locking member; and
delivering the flow of breathing gas to the patient through the patient sealing assembly.

10. (canceled)

11. The method according to claim 9, wherein the first and second proximal ends are passed through one or more holes provided in the central portion.

12. The method according to claim 9, wherein the sleeve member comprises a fabric material.

13. The method according to claim 9, wherein the cushion member comprises a nasal mask.

14. The method according to claim 13, wherein the cushion member comprises a nasal cushion.

15. The method according to claim 14, wherein the cushion member comprises a pillows style nasal cushion having first and second nasal prongs.
